(19) United States
(12) Patent Application Publication
(10) Pub. No.: US 2013/0102554 A1
(43) Pub. Date: Apr. 25, 2013

(54) COMPOSITION FOR TREATMENT OF OBESITY USING WHEAT BRAN EXTRACT OR ACTIVE INGREDIENT ISOLATED THEREFROM

(71) Applicants: Hai Soo Lee, Seoul (KR); Ok Pho Zee, Seoul (KR); Joa Sub Oh, Seoul (KR); Eun Kyung Ahn, Gyeonggi-do (KR); Jung A. Lee, Gyeonggi-do (KR); Seong Su Hong, Gyeonggi-do (KR)

(72) Inventors: Hai Soo Lee, Seoul (KR); Ok Pho Zee, Seoul (KR); Joa Sub Oh, Seoul (KR); Eun Kyung Ahn, Gyeonggi-do (KR); Jung A. Lee, Gyeonggi-do (KR); Seong Su Hong, Gyeonggi-do (KR)

(73) Assignee: HAI SOO LEE, Seoul (KR)

(21) Appl. No.: 13/520,161

(22) PCT Filed: Dec. 30, 2010
(86) PCT No.: PCT/KR2010/009592

§ 371 Date: Oct. 24, 2012

(30) Foreign Application Priority Data
Dec. 30, 2009 (KR) 10-2009-0134278
Aug. 11, 2010 (KR) 10-2010-0077286

(51) Int. Cl. A61K 31/7034; A61K 31/201

(52) U.S. Cl. CPC A61K 31/7034; A61K 31/201
USPC 514/35; 536/4.1; 554/223

(57) ABSTRACT

The present invention relates to a composition for treatment of obesity using a wheat bran extract or a tachioside or 9,12,13-trihydroxy-10(E)-octadecenoic acid, an active ingredient isolated therefrom. The wheat bran extract or the active ingredients inhibit the expression of PPARγ, C/EBRα and ADD1/SREBP1c, transcription factors which inhibit the differentiation of adipocyte progenitor cells to adipocytes, promoted by insulin, inhibit the accumulation of fats, and are centrally involved in the differentiation of adipocytes.

### TECHNICAL FIELD

The present invention relates to a composition for the improvement of obesity, comprising a wheat bran extract or an isolate produced from the extract as an active ingredient.

### BACKGROUND ART

Recently, the obese population in Korea has rapidly increased with the improvement in the standard of living, the lack of physical activity due to people being busy, and the intake of excessive nutrition. The Korean obese population has increased from 11.7% as of 1995 to 29.4% in 2001 for women and from 18.0% as of 1995 to 32.6% in 2001 for men (the Ministry of Health and Welfare of Korea, 2007).

Obesity is a condition in which adipose tissue increases abnormally with the accumulation of excessive energy due to an imbalance between energy intake and consumption (Clinical Endocrinology 28:675-689, 1998; Clinical Obesity (eds. Kopelman P G, Stock M J) 248-89 (Blackwell Science, Oxford 1998). Clinically, obesity is defined as a condition in which body fat accounts for more than 25% of the body weight for men and 30% for women. A person with a BMI (Body Mass Index) equal to or more than 30.0 is considered obese.

Obesity is caused by environmental factors such as a high-fat, high-energy diet, a lack of physical activity, endocrine disorders, and genetic factors. Environmental factors account for 50 to 70% of cases of obesity while the remainder is attributed to genetic susceptibility.

To achieve energy homeostasis in the body, triglycerides stored in adipocytes are hydrolyzed into free fatty acids and glycerol when energy is required. In contrast, excessive energy intake promotes the maturation of adipocytes and increases the accumulation of body fat, resulting in obesity.

Adipocytes differentiate from preadipocytes. Transcription factors including PPARγ (peroxisome proliferator-activated receptor γ), C/EBP family (CCAAT/enhancer binding proteins; C/EBRα, C/EBRβ and C/EBRδ) and ADD1/SREBP1c (adipocyte determination differentiation factor 1)/(sterol regulatory element binding protein 1c) play a crucial role in adipocyte differentiation (Genes De 2000, 14(11) 1293˜1307). The expression of these transcription factors is induced at different points of time in the course of adipocyte differentiation and they interact with one another to regulate adipocyte-specific genes and progressively induce fat metabolism and adipocyte differentiation (Physiol Rev 1998, 78(3):783˜809; Annu Rev Biochem 2008, 77:289˜312; Genes Dev 1996, 10:1096˜1107).

Accordingly, a material that has the activity of regulating differentiation from preadipocytes to adipocytes and/or fat accumulation in adipose tissues or suppressing the expression of PPARγ, C/EBRα, and/or ADD1/SREBP1c may be used as an anti-obesity agent. As such, ginkgolide A or bilobalide is disclosed in Korean Patent No. 920648, ginsenoside-Rf or compound K in Korean Patent No. 847252, and oltipraz in Korean Patent No. 576157.

Culminating in the present invention, intensive and through research into the treatment of obesity led to the finding that a wheat bran extract or isolates produced from the extract can inhibit adipocyte differentiation and fat accumulation and suppress the expression of PPARγ, C/EBRα, and ADD1/SREBP1c.

### DISCLOSURE

### Technical Problem

It is therefore an object of the present invention to provide a composition for the improvement of obesity, comprising a wheat bran extract or an isolate produced from the extract as an active ingredient.

Other technical features will be apparent from the following description.

### Technical Solution

As will be explained in detail in the following Example section, a wheat bran extract or isolates from the extract, identified as tachioside (methoxy-hydroquinone-4-β-D-glucopyranoside) and 9,12,13-trihydroxy-10(E)-octadecenoic acid, are found to regulate insulin-induced adipocyte differentiation from preadiopcytes and fat accumulation and to suppress the expression of PPARγ, C/EBRα and ADD1/SREBP1c, which are transcription factors playing a pivotal role in adipocyte differentiation.

Tachioside can be separated from sugarcane molasses, Berchemia racemosa, and bamboo culm [J. Agr. Food Chem. 31: 545-548 (1983); Phytochemistry 26: 2811-2814 (1987); Food Sci. Biotechnol. 17(6):1376-1378 (2008)]. 9,12,13-Trihydroxy-10(E)-octadecenoic acid is found in licorice and Colocasia antiquorum [Izv Akad Nauk SSSRBiol. 6:932-6 (1988); Phytochemistry 28: 2613-2615 (1989)].

The compounds useful as an active ingredient in the present invention have the following IUPAC names and chemical formulae, respectively.

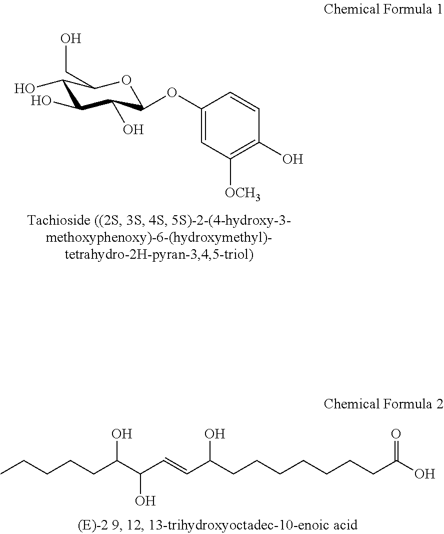

In accordance with an aspect thereof, the present invention provides a composition for the improvement of obesity, comprising a wheat bran extract or the compound of Chemical Formula 1 and/or the compound of Chemical Formula 2 as an active ingredient.

The term “wheat bran,” as used herein, is intended to refer to all of the byproducts produced upon the polishing of wheat. The byproducts may comprise wheat with the exclusion of the endosperm (which is a material making up wheat flour), or may be composed mainly of the wheat hull when the embryo is separated together with the endosperm. Depending on the degree of polishing, the wheat bran may comprise a small amount of endosperm and embryo. Wheat bran may be further milled into bran powder which is further categorized depending on the degree of polishing. The term “wheat bran” also includes the powder resulting from polishing wheat bran.

The term “extract,” as used herein, is intended to include not only a crude extract produced from wheat bran, by use of a solvent selected from among water, lower alcohols of 1 to 4 carbon atoms, such as methanol, ethanol, butanol, etc., ethylene, acetone, hexane, ether, chloroform, ethylacetate, butylacetate, dichloromethane, N,N-dimethylformamide (DMF), dimethylsulfoxide (DMSO), 1,3-butylene glycol, propylene glycol and a combination thereof, but also a fraction of the crude extract in such a solvent. So long as it assures the extraction and preservation of the active ingredient, any extraction method may be employed. Examples of the extraction method include cold precipitation, refluxing, warming, and ultrasonication. The fraction includes those obtained by partitioning the crude extract between two solvents which have different polarities and eluates obtained by eluting the crude extract loaded into a silica gel-filled column using a hydrophobic solvent, a hydrophilic solvent or a combination thereof as a mobile phase. In addition, the extract of the present invention may be in a concentrated liquid phase or a solid phase as a result of removing the extraction solvent by freeze drying, vacuum drying, hot-air drying, or spray drying. Preferably, the extract of the present invention may be a crude extract produced from wheat bran using a solvent selected from the group consisting of water, ethanol and a combination thereof, or a fraction of the crude extract.

As used herein, the term “obesity” means an abnormal increase of adipose tissue, whether caused by a genetic factor or an environmental factor, and is intended to include both obesity and overweight as defined by the BMI standard (a BMI of 30.0 or greater for obesity and a BMI of 25˜30 for overweight).

As used herein, the term “active ingredient” means a component that can exhibit the desired activity, alone or in combination with a vehicle, which is itself not active.

The term “improvement,” as used herein in connection with obesity, is intended to include the prevention or treatment of obesity and body fact reduction and/or weight loss.

So long as it assures the improvement of obesity, any amount (effective amount) of the active ingredient may be used in the composition of the present invention. Depending on the use, formulation, and purpose of the composition, the effective amount varies typically within the range from 0.001 weight % to 99.990 weight % based on the total weight of the composition. The term “effective amount,” as used herein, means a dosage of the active agent sufficient to produce a desired result, for instance inducing the amelioration and treatment of obesity. The effective amount may be experimentally determined within the ordinary ability of those skilled in the art.

The subjects to which the composition of the present invention is applied are animals and humans, and preferably humans.

In accordance with another aspect thereof, the present invention provides a composition for dieting, comprising a wheat bran extract or the compound of Chemical Formula 1 and/or the compound of Chemical Formula 2 as an active ingredient.

As used herein, the term “diet” is intended to refer to a condition in which a reduction in the body weight/body fat is desired or necessary for the purpose of aesthetics or health although unrelated to being either overweight or obese. Generally, the diet composition of the present invention may be prepared for normal people for the sake of aesthetics or health.

In connection with the wheat bran extract and its effective amount, a description given to the composition for the improvement of obesity is true of the composition for dieting.

In accordance with a further aspect thereof, the present invention provides a composition for the improvement of insulin resistance, comprising a wheat bran extract or the compound of Chemical Formula 1 and/or the compound of Chemical Formula 2 as an active ingredient.

Obesity is one of many causes of insulin resistance. Although persons with severe obesity may have no insulin resistance, there is a close correlation between insulin resistance and obesity. On the whole, it is known that an increase in the severity of obesity, particularly visceral obesity, increases the insulin resistance.

Adipocytes release adipocytokines, which play an important role in the maintenance of metabolism homeostasis. Obesity, that is, excessive fat accumulation induces the hyper- or hypo-production of adipocytokines so that the homeostasis is disrupted, incurring insulin resistance.

Adipocytokines may increase insulin sensitivity and cause insulin resistance. Representative among the former are adiponectin, leptin and AMPK (AMP-dependent protein kinase) while the latter includes TNF-α, IL-6, and resistin.

Also, Fas (fatty acid synthase) or aP2 (adipocyte fatty-acid-binding protein 2), which are both expressed in adipocytes, are known to be involved in insulin resistance.

Fas is expressed in an early stage of adipocyte differentiation (J. Biol. Chem., 255:4745˜4750 (1980)) and functions to catalyze the synthesis of palmitate from acetyl-CoA and malonyl-CoA. Fas plays a role in the storage of surplus energy in the form of triglycerides, which may cause obesity. The resulting triglyceride palmitate destroys pancreatic 0 cells to induce insulin resistance (Proc. Natl. Acad. Sci. 95:2498˜2502 (1998)). Based on the report that insulin resistance occurs at low rates in aP2-deficient mice and an aP2 inhibitor reduces insulin resistance, aP2 is suggested as a promising target useful for developing therapeutics for insulin resistance (Nature 447:959-965 (2007)).

To examine whether the compounds of Chemical Formulas 1 and 2, which are the active ingredients for improving obesity, can be used as anti-insulin resistance agents, they were applied to adipocytes which were then quantitatively analyzed for resistin, a lipocytokine involved in insulin resistance, and Fas and aP2. Their expression levels were significantly suppressed. These experimental data demonstrate that the compounds of Chemical Formulas 1 and 2 can be effectively used to improve insulin resistance as well as obesity.

As used herein, the term “insulin resistance” refers to a physiological condition in which more insulin than is used typically is required for the operation of the normal metabolism of cells, organs and the body, that is, the condition of insulin dysfunction in which insulin is less effective, and is considered the equivalent of insulin-non-dependent diabetes or type 2 diabetes. Diabetes are classified into insulin-dependent diabetes (type 1 diabetes), characterized by the loss of pancreatic β-cells, and insulin-non-dependent diabetes (type 2 diabetes), characterized by insulin resistance. Accordingly, insulin resistance, insulin-non-dependent diabetes and type 2 diabetes are considered the same in the art.

With regard to the wheat bran extract and its effective amount, a description given of the composition for the improvement of obesity is true of the composition for the improvement of insulin resistance.

In one preferred embodiment, the composition of the present invention may be used as a food composition.

The food composition of the present invention may be for health aid foods, nutrient supplements, or functional beverages.

The food composition may contain an additive such as a sweetener, a flavoring agent, a physiologically active substance, a mineral, etc. in addition to the active ingredient.

A sweetener is used to impart a sweet taste to the composition and may be natural or synthetic. Preferable is a natural sweetener. Examples of the natural sweetener include corn syrup, honey, sucrose, fructose, lactose, maltose and other sugars.

A flavoring agent is adopted to enhance the taste or flavor of the composition and may be natural or synthetic. Preferable is a natural flavoring agent. A flavoring agent, if natural, may have the function of nutritional supplementation in addition to enhancing the flavor. Examples of the natural flavoring agent include those obtained from apple, lemon, mandarin, grape, strawberry, peach, green tea leaves, Polygonatum odoratum, bamboo leaves, cinnamon, chrysanthemum leaves, and/or jasmine. Other natural flavoring agents include those from ginseng (red ginseng), bamboo shoots, aloe vera, and ginkgo nuts. The natural flavoring agent may be in the form of a liquid concentrate or a solid extract. A synthetic flavoring agent may be used, and is exemplified by esters, alcohols, aldehydes and terpenes.

Among the physiological active substance are catechins, such as catechin, epicatechin, gallocatechin and epigallocatechin, and vitamins, such as retinols, ascorbic acid, tocopherol, calciferol, thiamine and rivoflavin.

As for the mineral, examples thereof include calcium, magnesium, chrome, cobalt, copper, fluorides, germanium, iodine, iron, lithium, magnesium, manganese, molybdenum, phosphorus, calcium, selenium, silicone, sodium, sulfur, vanadium, and zinc.

Optionally, the food composition of the present invention may further comprise a preservative, an emulsifier, an acidulant, and a thickener in addition to additives such as a sweetener, etc.

The agents such as preservatives, emulsifiers, etc. are used in as minimal an amount as possible to achieve the purpose of their addition. Numerically, their amount ranges from approximately 0.0005% by weight to 0.5% by weight based on the total weight of the composition.

Examples of the preservative useful in the present invention include calcium sorbate, sodium sorbate, potassium sorbate, calcium benzoate, sodium benzoate, potassium benzoate, and EDTA (ethylenediaminetetracetic acid).

Acacia gum, carboxymethylcellulose, xanthan gum, and pectin are emulsifiers that can be used in the present invention.

Representative among the acidulants are citric acid, malic acid, fumaric acid, adipic acid, phosphoric acid, gluconic acid, tartaric acid, ascorbic acid and acetic acid. The acidulant may be added for the purpose of suppressing microbial proliferation as well as enhancing the taste.

The thickener useful in the present invention may be exemplified by a suspending agent, a flocculant, a gel forming agent, and a swelling agent.

The food composition of the present invention may further comprise a natural additive to enhance the taste or flavor or an agent known as having anti-obesity activity or as a liver function enhancer (to improve a fatty liver or to treat a hangover). Examples of such an additive or agent include clotted cow blood powder or extract, a bean sprout powder or extract, a shellfish powder or extract, an oyster powder or extract, a Cnidium monnieri powder or extract, a radish juice or extract, a cucumber juice or extract, a Chinese chive juice or extract, a spinach juice or extract, a lotus root juice or extract, a kuzu vine juice or extract, a pine needle juice or extract, a ginseng juice or extract, a Hedyotis diffusa powder or extract, licorice powder or extract, a Pueraria lobata flower powder or extract, a Pueraria lobata root powder or extract, a Amomum villosum LOUR powder or extract, a gourd powder or extract, a ginger powder or extract, a jujube powder or extract, a Artemisia capillaris Thunb.) powder or extract, a Hovenia dulcis Thunb seed powder or extract, a Silybum marianum powder or extract, a Atractylodes macrocephala Koidzumi powder or extract, a Polyporus umbellatus Fries powder or extract, a Citrus unshiu Markovich peel powder or extract, a Lycium chinense Miller powder or extract, a green tea powder or extract, a Schisandra chinensis powder or extract, an oriental raisin tree extract, a Lithospermum erythrorhizon S. et Z extract, a Phragmites communis Trinius extract, a cinnamon extract, and decursinol. With regard to the meaning of “extract”, the description give to the wheat bran extract may be applied. The extracts may be combined in a mixture.

In another preferred embodiment, the composition of the present invention may be used as a pharmaceutical composition.

The pharmaceutical composition according to the present invention comprises a pharmaceutically acceptable vehicle or excipient in addition to the active ingredient and may be formulated into oral dosage forms (tablets, suspensions, granules, emulsions, capsules, syrup, etc.), parenteral dosage forms (sterile injections, aqueous or oily suspensions, etc.), and topical application forms (solutions, creams, ointments, gels, lotions, patches, etc.).

As used herein, the term “pharmaceutically acceptable” means that a material does not interfere with the effectiveness of the biological activity of the active ingredients and is low enough in toxicity to be used on the subject.

Examples of the pharmaceutically acceptable vehicle include lactose, glucose, sucrose, starch (e.g., corn starch, potato starch, etc.), cellulose and its derivatives (e.g., sodium carboxymethyl cellulose, ethyl cellulose, etc.), malt, gelatin, talc, a solid lubricant (e.g., stearic acid, magnesium stearate, etc.), calcium sulfate, vegetable oil (e.g., peanut oil, cotton seed oil, sesame oil, olive oil), polyol (e.g., propylene glycol, glycerine), alginic acid, emulsifiers (e.g., TWEENS), wetting agents (sodium lauryl sulfate), colorants, flavoring agents, stabilizers, antioxidants, preservatives, water, saline, and phosphate buffered saline. These vehicles may be used, individually or in combination, according to the formulation of the pharmaceutical composition.

A suitable excipient may also be employed in the pharmaceutical composition of the present invention. For example, an excipient suitable for formulating the pharmaceutical composition of the present invention into an aqueous suspension may be a suspending agent or dispersant such as sodium carboxymethyl cellulose, methyl cellulose, hydropropylmethylcellulose, sodium alginate, or polyvinylpyrrolidone. When the pharmaceutical composition is formulated into an injection, Ringer's solution, or isotonic sodium chloride may be used as an excipient.

To administer the pharmaceutical composition of the present invention, an oral route or a parenteral route such as a topical route may be taken.

The daily dose pharmaceutically composition of the present invention may be administered at a daily dose of from 0.001˜150 mg/kg of body weight and in a single dose or in multiple doses per day. The dose of the pharmaceutical composition of the present invention may vary depending on various factors including the route of administration, the patient's age, gender and weight, and the severity of illness and thus must be in no way understood as limiting the scope of the present invention.

### Advantageous Effects

As described hitherto, a composition comprising a wheat bran extract or the compound of Chemical Formula 1 or 2 is provided for improving obesity. Also, the present invention provides a composition for the improvement of insulin resistance. The composition for the treatment of obesity or insulin resistance according to the present invention may be used as a functional food composition or a pharmaceutical composition.

### DESCRIPTION OF DRAWINGS

FIG. 1 is a schematic diagram showing the isolation of compounds 1 and 2.

FIG. 2 is a COSY spectrum of the compound of Chemical Formula 2.

FIGS. 3 to 8 show inhibitory activities of the wheat bran extract and the compounds of Chemical Formulas 1 and 2 against lipid accumulation of adipocytes in dose-dependent manners.

FIGS. 9 and 10 show inhibitory activities of the wheat bran extract and the compounds of Chemical Formulas 1 and 2 against PPARγ transcription activity in dose-dependent manners.

FIGS. 11 to 16 show inhibitory activities of the wheat bran extract and the compounds of Chemical Formulas 1 and 2 against the expression of PPARγ, C/EBRα and ADD1/SREBP1c in dose-dependent manners at the genetic level.

FIGS. 17 and 18 show inhibitory activities of the compounds of Chemical Formulas 1 and 2 against the expression of PPARγ and C/EBRα in dose-dependent manners at a protein level.

FIGS. 19 to 24 show inhibitory activities of the compounds of Chemical Formulas 1 and 2 against the expression of resistin, aP2 and Fas in dose-dependent manners at the genetic level.

### MODE FOR INVENTION

A better understanding of the present invention may be obtained through the following examples which are set forth to illustrate, but are not to be construed as limiting the present invention.

### EXAMPLES

### Preparation of Wheat Bran Extract and Isolation and Identification of Active Ingredients

### Example 1

### Preparation of Wheat Bran Extract 1

Hot water extraction was performed for 6 hours using 200 g of wheat bran, which was the remainder left after polishing wheat (Triticum aestivum L.), in 2 L of water, followed by filtration. The filtrate was loaded to a column filled with Diaion HP-20 resins and then eluted using 100% ethanol and water as mobile phases. The eluate was named Red-dog A for the 100% ethanol fraction and Red-dog B for the water fraction.

### Example 2

### Preparation of Wheat Bran Extract 2

Ethanol was added to wheat bran, which was the remainder after the polishing of wheat (Triticum aestivum L.), followed by extraction by three rounds of cold precipitation for 24 hours. After the filtration of the extract, the filtrate was concentrated in a vacuum. The concentrate was suspended in distilled water and fractioned with CH₂Cl₂. The aqueous layer was partitioned again with butanol. The butanol fraction (G36W) was evaporated in a vacuum and subjected to Diaion HP-20 column chromatography using a mixture of water-methanol (water, 20, 40, 60, 80, 100% methanol) as a mobile phase to produce six sub-fractions (G36W-18-1˜6).

### Example 3

### Isolation of Active Ingredients from Wheat Bran Extract

The sub-fraction G36W-18-2 of Example 2 was subjected to silica gel column chromatography (10×25) using CHCl₃:MeOH:H₂O (20:4:1, 10:3:1, 6:3:1, 6:4:1) as a mobile phase to give 11 sub-fractions (G36W-20-1˜11). Compound 1 was isolated from the sub-fraction G36W-20-5. The sub-fraction G36W-18-5 was partitioned between CH₂Cl₂ and water, after which re-crystallization of the CH₂Cl₂ layer afforded Compound 2.

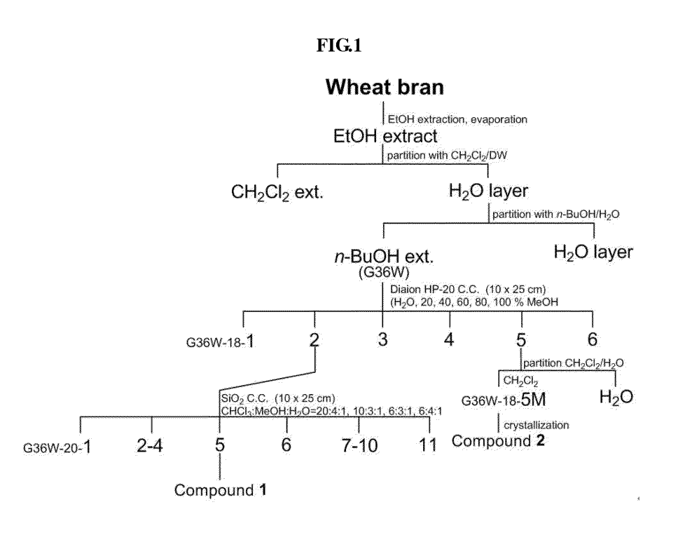

The isolation scheme of the active ingredients is shown in FIG. 1.

### Example 4

### Identification of the Active Ingredient Compounds 1 and 2 Isolated from the Wheat Bran Extract

### Example 4-1

### Identification of Compound 1

Physicochemical and spectral analysis was conducted on the isolated compound 1.

Colorless needles; ESIMS (positive mode) m/z 325.35 [M+Na]⁺, 627.35 [2M+Na]⁺; (negative mode) m/z 301.79 [M+H]⁻, 603.20 [2M+H]⁻;

¹H-NMR (DMSO-d₆, 400 MHz) δ 6.69 (1H, d, J=2.8 Hz, H-3), 6.66 (1H, d, J=8.8 Hz, H-6), 6.46 (1H, dd, J=8.8, 2.8 Hz, H-5), 4.67 (1H, d, J=7.6 Hz, H-1′, 3.73 (3H, s, 3-OCH3), 3.71 (1H, dd, J=11.6, 4.8 Hz, H-6′), 3.44 (1H, dd, J=11.6, 6.0 Hz, H-6′), 93.10-3.45 (4H, m, H-2′,3′,4′,5′);

¹³C-NMR (DMSO-d₆, 100 MHz) δ 151.2 (C-4), 148.2 (C-2), 141.7 (C-1), 115.6 (C-6), 108.3 (C-5), 102.8 (C-3), 102.1 (C-1′), 77.5 (C-3′), 77.2 (C-5′), 73.7 (C-2′), 70.4 (C-4′), 61.3 (C-6′), 55.9 (OCH₃).

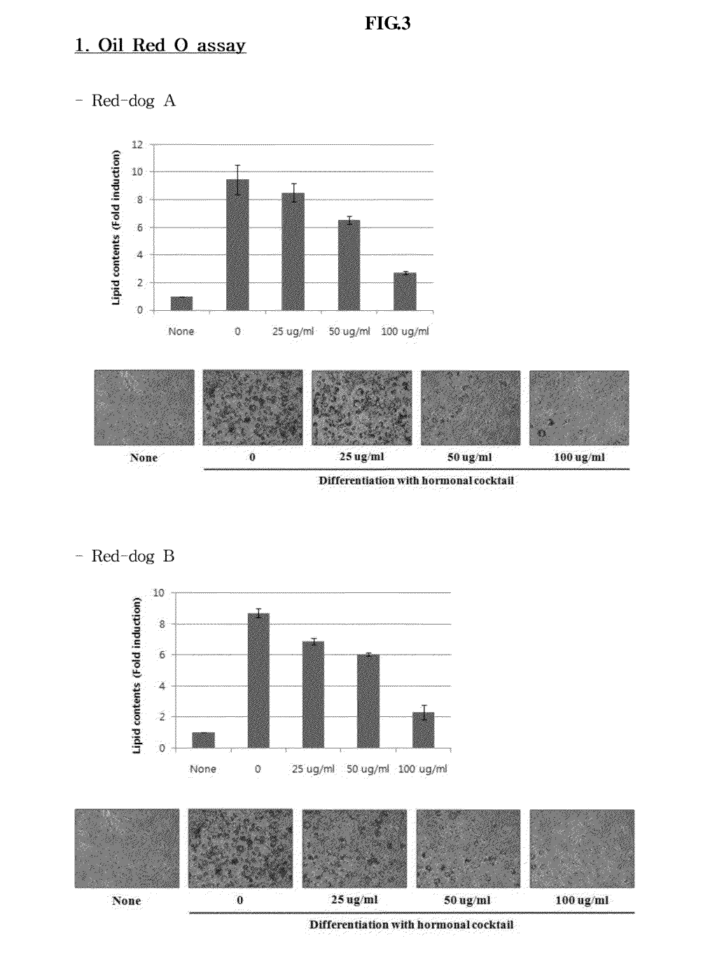

Compound 1 was obtained as a colorless needle-form crystal and found to have a molecular weight of 302 amu as measured by ESI-MS (m/z 325.35 [M+Na]⁺, 301.79 [M+H]⁻). Its molecular formula was identified as C₁₃H₁₈O₈ with a degree of unsaturation of 5. In a ¹H-NMR spectrum (FIG. 3), 1,2,4-trisubstituted aromatic protons [δ_{H} 6.69 (1H, d, J=2.8 Hz), 6.66 (1H, d, J=8.8 Hz), 6.46 (1H, dd, J=8.8, 2.8 Hz)] were observed and signals attributed to one methoxy group [δ_{H} 3.73 (3H, s)] and one anomeric proton attributed to glucose at δ 4.67 (1H, d, J=7.6 Hz) were identified. A ¹³C-NMR read a total of 13 carbon signals inclusive of six benzene ring signals, six sugar signals [δ_{C} 102.1 (C-1′), 77.5 (C-3′), 77.2 (C-5′), 73.7 (C-2′), 70.4 (C-4′), 61.3 (C-6′)], and one methoxy signal at δ_{C} 55.9.

From the spectral data and the degree of unsaturation, compound 1 was inferred to be a compound in which a glucose moiety is linked to a methoxyhydroquinone moiety. Physicochemical properties and consultation with references (Food Sci. Biotechnol., 17(6), 1376-1378, Tachioside, an antioxidative phenolic glycoside from bamboo Species) identified compound 1 as tachioside (methoxy-hydroquinone-4-β-D-glucopyranoside) of Chemical Formula 1.

### Example 4-2

### Identification of Compound 2

Physicochemical and spectral analysis was conducted on the isolated compound 1.

White solid; ESIMS (positive mode) m/z 353.85 [M+Na]⁺, 683.42 [2M+Na]⁺; (negative mode) m/z 329.72 [M+H]⁻, 659.83 [2M+H]⁻;

¹H-NMR (CDCl₃/CD₃OD, 400 MHz) δ 5.73 (1H, dd, J=15.6, 6.0 Hz, H-10), 5.65 (1H, dd, J=15.6, 6.4 Hz, H-11), 4.06 (1H, dd, J=12.4, 6.5 Hz, H-9), 3.89 (1H, t, J=6.4 Hz, H-12), 3.41 (1H, m, H-13), 2.28 (2H, t, J=7.6 Hz, H-2), 1.61 (2H, m, H-3), 1.52 (2H, m, H-8a, 14a), 1.32 (16H, m, H-4, 5, 6, 7, 8b, 14b, 15, 16, 17), 0.89 (3H, t, J=7.0 Hz, H-18);

¹H-NMR (pyridine-d5, 400 MHz) δ 6.42 (1H, dd, J=15.6, 5.6 Hz), 6.35 (1H, dd, J=15.6, 5.2 Hz), 4.53 (2H, m), 3.96 (1H, m), 2.51 (2H, t, J=7.4 Hz), 1.81 (7H, m), 1.58 (3H, m), 1.33 (10H, m), 0.83 (3H, t, J=6.8 Hz);

¹³C-NMR (pyridine-d5, 100 MHz) δ 177.0, 137.6, 131.8, 77.2, 76.2, 72.8, 39.4, 35.8, 34.5, 33.3, 30.9, 30.7, 30.5, 27.2, 27.0, 26.6, 23.9, 15.2.

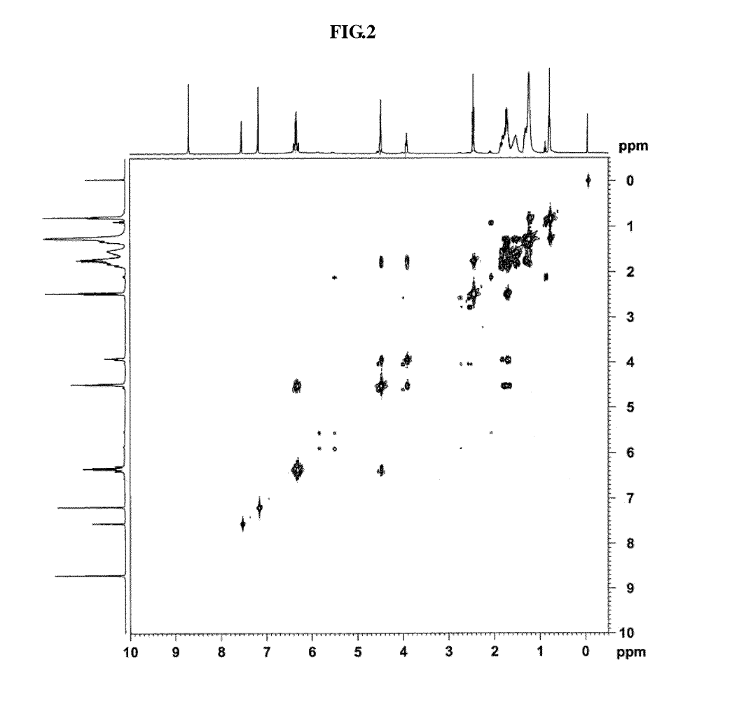

Compound 1 was obtained as a white solid and found to have a molecular weight of 330 amu as measured by ESI-MS (m/z 353.85 [M+Na]⁺, 329.72 [M+H]⁻). Its molecular formula was identified as C₁₈H₃₄O₅ with a degree of unsaturation of 2. In ¹H- and ¹³C-NMR, three oxygenated methine protons were detected respectively at δ_{H} 4.06 (1H, dd, J=12.4, 6.5 Hz, H-9), 3.89 (1H, t, J=6.4 Hz, H-12), and 3.41 (1H, m, H-13) and signals attributed to one trans double bond appeared [δ_{H} 5.73 (1H, dd, J=15.6, 6.0 Hz; δ_{C} 131.8), 5.65 (1H, dd, J=15.6, 6.4 Hz; δ_{C} 137.6)]. Also, a carboxyl carbon signal was detected at δ_{C} 177.0. Thus, compound 2 was inferred to have a monounsaturated long-chain fatty acid structure of 18 carbon atoms with three hydroxy groups and one trans double bond. In addition, COSY (see FIG. 2) revealed the partial structure of CH₂—CH(OH)—CH(OH)—CH═CH—CH(OH)—CH₂—. The data obtained above and consultation with references (J. Nat. Prod., 2006 69(9), 1366-1369, Phytochemicalconstituents from Salsola tetranda) identified compound 2 as 9,12,13-trihydroxy-10(E)-octadecenoic acid of Chemical Formula 2.

### EXPERIMENTAL EXAMPLES

### Assays of Wheat Bran Extract and Isolates Therefrom for Inhibitory Activity Against Obesity and Insulin Resistance

### Experimental Example 1

### Assay for Activity of Improving Obesity

The mouse preadipocyte 3T3-L1 was cultured at 37° C. in 10% BCS DMEM under a 5% CO₂ condition.

3T3-L1 preadipocytes were seeded at a density of 5×10⁴ cells/well into 24-well plates. After being grown to 100% confluency, the cells were incubated for an additional two days. Then, the preadipocytes were induced to differentiate into adipcytes in the presence of MDI (0.5 mM 3-isobutyl-1-methylxanthine (IBMX), 1 μM dexamethasone, 1 μg/ml insulin) in 10% FBS DMEM. After incubation for 48 hours in this medium, the cells were cultured for two days in 10% FBS DMEM containing 1 μg/ml insulin. Subsequently, the cells were cultured for four days in 10% FBS DMEM, with replacement of the medium with a fresh one every two days. During adipocyte differentiation, the cells were treated with predetermined concentrations of the samples. On day 8 at which differentiation was completed, observations were made of the differentiation of the cells. In this connection, the adipose differentiation was qualitatively analyzed with Oil Red 0 staining under an optical microscope and quantitatively analyzed by measuring absorbance at 510 nm.

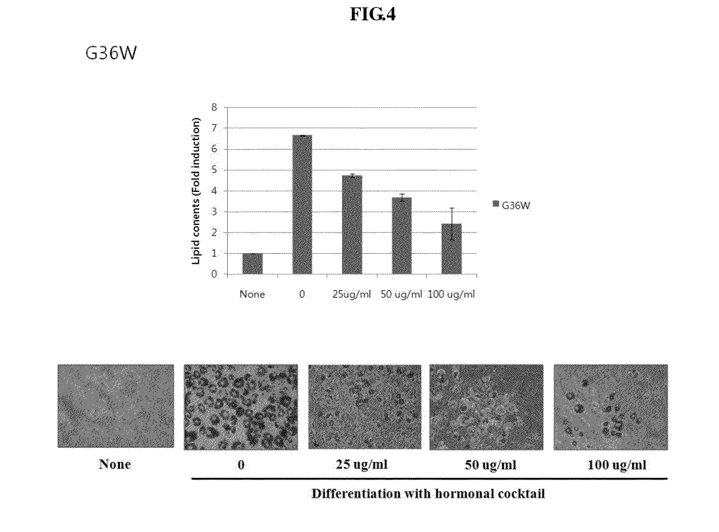

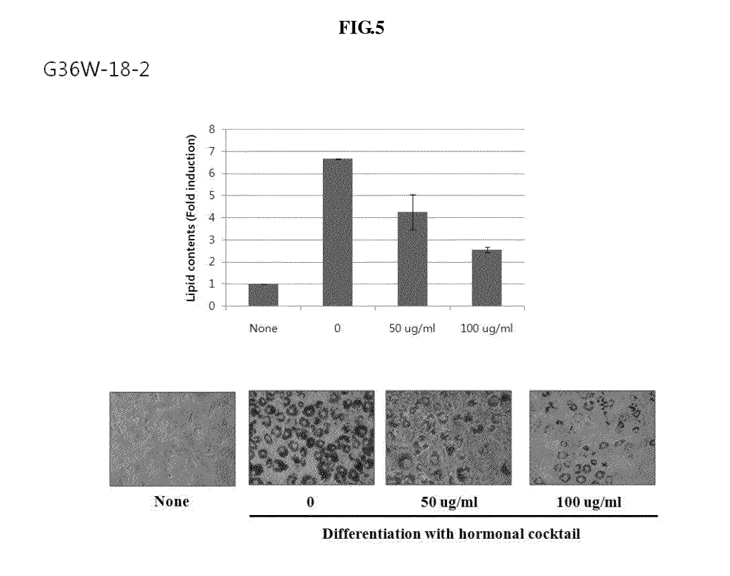

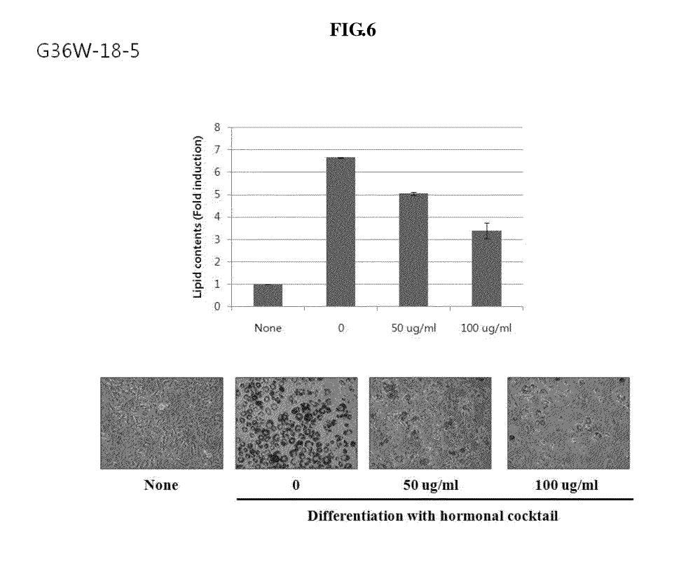

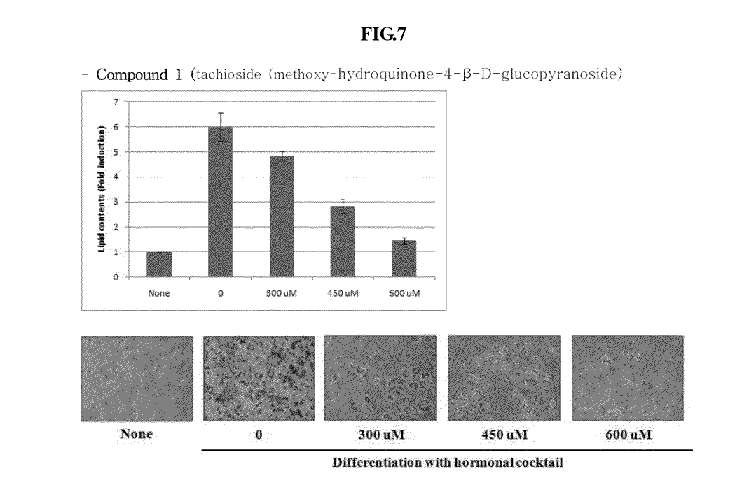

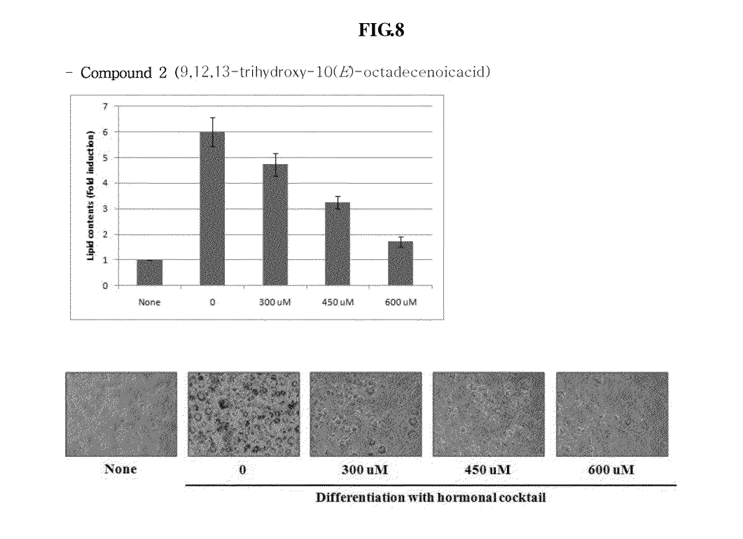

The results are shown in FIGS. 3 to 8. FIGS. 3 to 8 show Oil Red O staining and lipid contents in cells treated with the extracts of Example 1 (Red-dog A & Red-dog B) and Example 2 (G36W, G36W-18-2 & G36W-18-5), and the compounds of Example 3 (compounds 1 & 2).

As can be seen in FIGS. 3 to 8, all the wheat bran extract and compounds 1 and 2 produced from the extract inhibited the differentiation of preadipocytes into adipocytes in dose-dependent manners.

### Experimental Example 1-2

### Assay for Inhibitory Activity Against the Transcriptional Factor PPARγ

HEK 293T cells were co-transfected with a recombinant pGL3-basic luciferase expression vector (Promega) carrying a PPRE (PPARγ response element) gene and a PPARγ gene and a pRL-SV-40 plasmid (Promega) carrying a Renilla luciferase cDNA as a control reporter. One day after transfection, the cells were treated for 24 hours with predetermined concentrations of the samples (Red-dog A, Red-dog B and Compounds 1 & 2) alone or in combination with 10 μM troglitazone, a ligand to PPARγ. The inhibitory activity of the samples against the transcription factor PPARγ was determined by measuring the expression levels of luciferase.

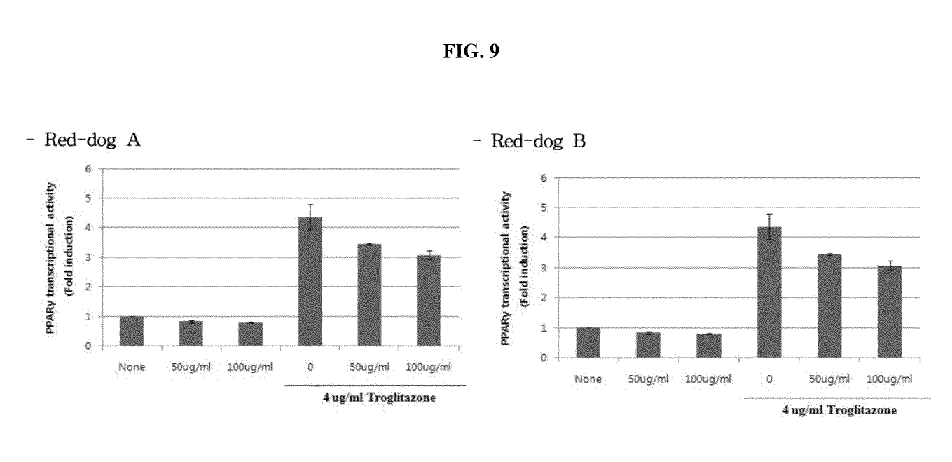

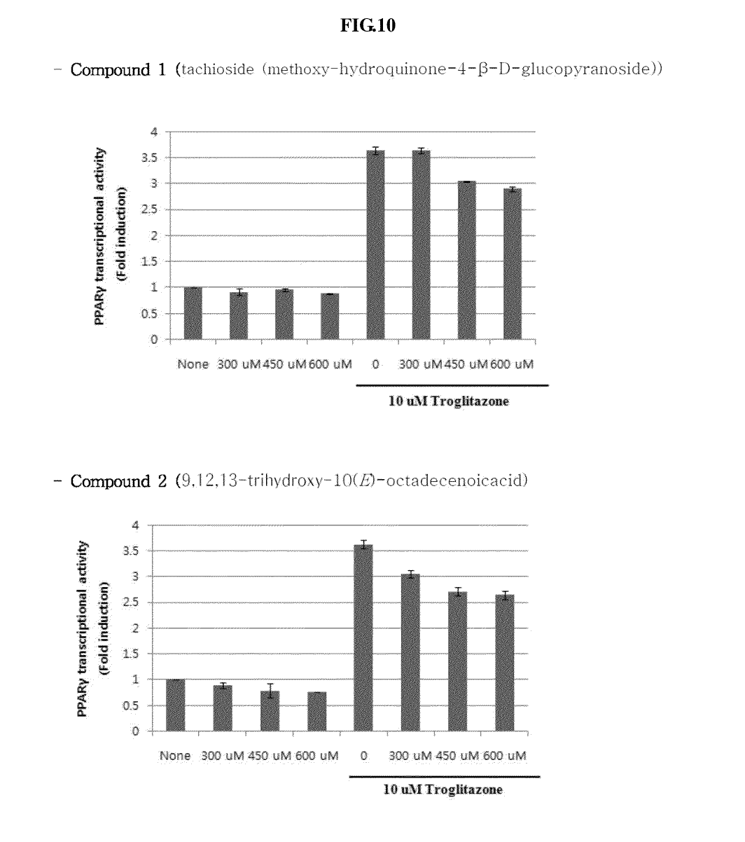

The results are shown in FIGS. 9 and 10. FIGS. 9 and 10 show the effects of the samples, that is, the extracts of Example 1 (Red-dog A & Red-dog B) and the compounds of Example 3 (Compounds 1 & 2), on the transcriptional activity of PPARγ. As can be seen in FIGS. 9 and 10, all of the samples inhibited the transcriptional activity of PPARγ in a dose-dependent manner.

### Experimental Example 1-3

### Inhibitory Activity Against Transcription Factors Responsible for Adipocyte Differentiation

### Experimental Example 1-3-1

### Inhibitory Activity Assay at Gene Level

During adipocyte differentiation, expression levels of PPARγ, C/EBRα and ADD1/SREBP1c are increased.

After being treated with predetermined concentrations of the samples (Red-dog A, Red-dog B and Compounds 1 & 2) in the same manner as in Experimental Example 1-2, the cells were induced to differentiate into adipocytes for 8 days. The expression levels of PPARγ, C/EBRα and ADD1/SREBP1c were analyzed using real-time PCR. The 3T3-L1 cells were washed twice with PBS after differentiation for 8 days and the cell pellets were used for RNA isolation using an RNA prep kit (Qiagen). cDNA was synthesized from 1 μg of the isolated RNA before real-time PCR using SYBR green (Takara) and the primers given in Table 1, below. As a control gene, GAPDH was used. In this regard, the cDNA synthesized from 1 μg of RNA was diluted 1/50 to a volume of 5 μL and mixed with 0.5 μL of each of 10 pmole primer, 10 μL of 2XSYBR green, and 4 μL of distilled water to give a PCR mix with a total volume of 20 μL. Real-time PCR started with denaturation at 95° C. for 30 sec, followed by 40 thermal cycles of denaturation at 95° C. for 5 sec, annealing for 60° C. for 15 sec and extension at 72° C. for 10 sec. The melt curve consisted of 80 melt cycles, starting at 55° C. to 95° C. with increments of 0.5° C. per cycle. Desired fluorescent signals were detected (data analysis was done with Bio-Rad MyiQ program).

TABLE 1
Primer

| Gene name | Forward primer | Reverse primer |
|---|---|---|
| GAPDH | GAGTCAACGGATT | GACAAGCTTCCCGT |
| | TGGTCGT | TCTCAG |
| | (SEQ ID NO: 1) | (SEQ ID NO: 2) |
| PPARγ | CGCTGATGCACTG | AGAGGTCCACAGAG |
| | CCTATGA | CTGATTCC |
| | (SEQ ID NO: 3) | (SEQ ID NO: 4) |
| C/EBPα | AGGTGCTGGAGTT | CAGCCTAGAGATCC |
| | GACCAGT | AGCGAC |
| | (SEQ ID NO: 5) | (SEQ ID NO: 6) |
| ADD1/ | CAAACTGCCCATC | TGCCTCCTCCACTG |
| SREBP1c | CACCGAC | CCACAA |
| | (SEQ ID NO: 7) | (SEQ ID NO: 8) |

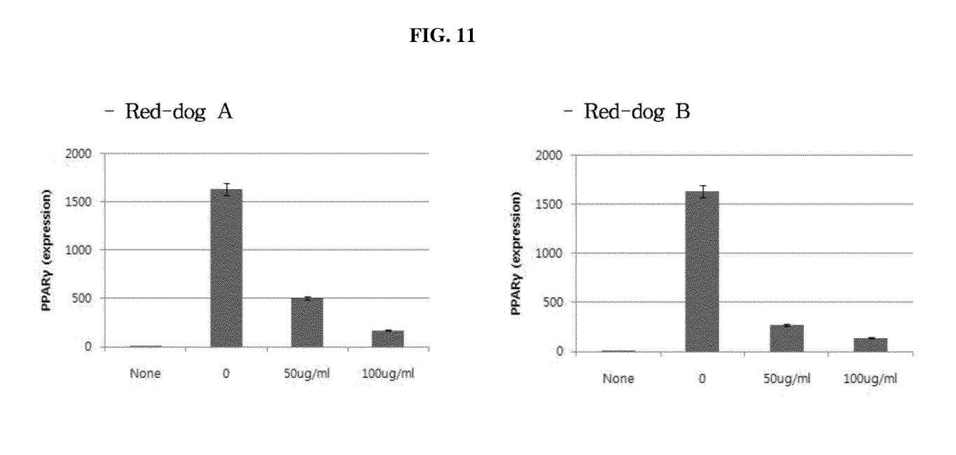

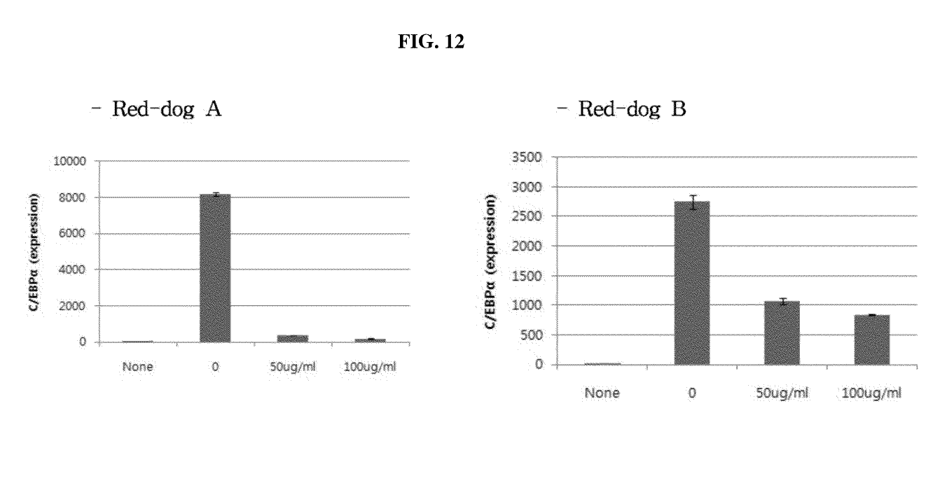

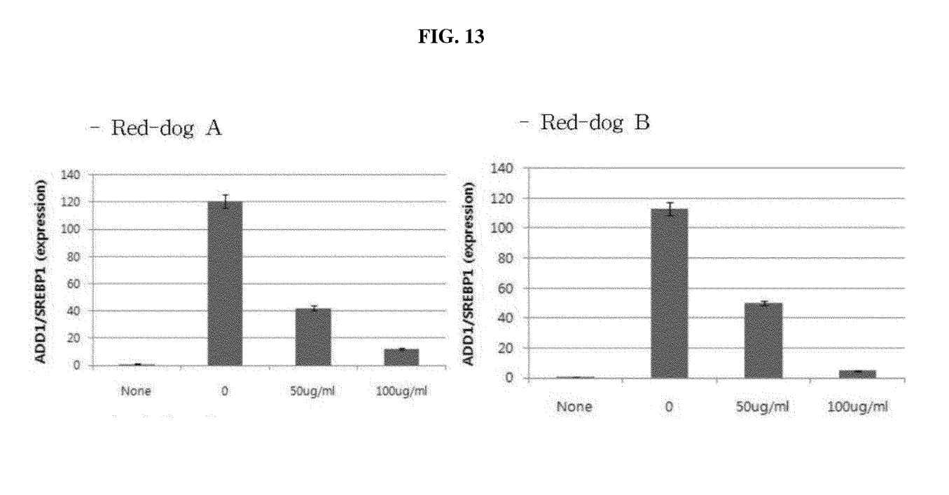

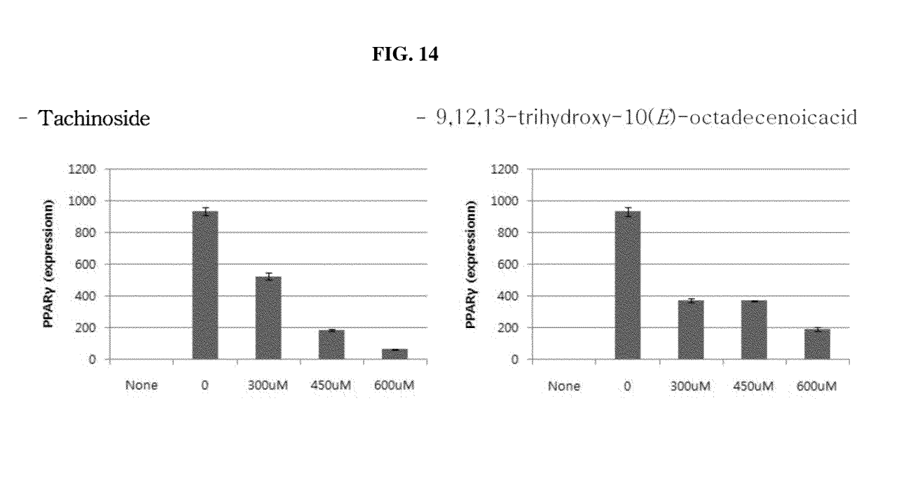

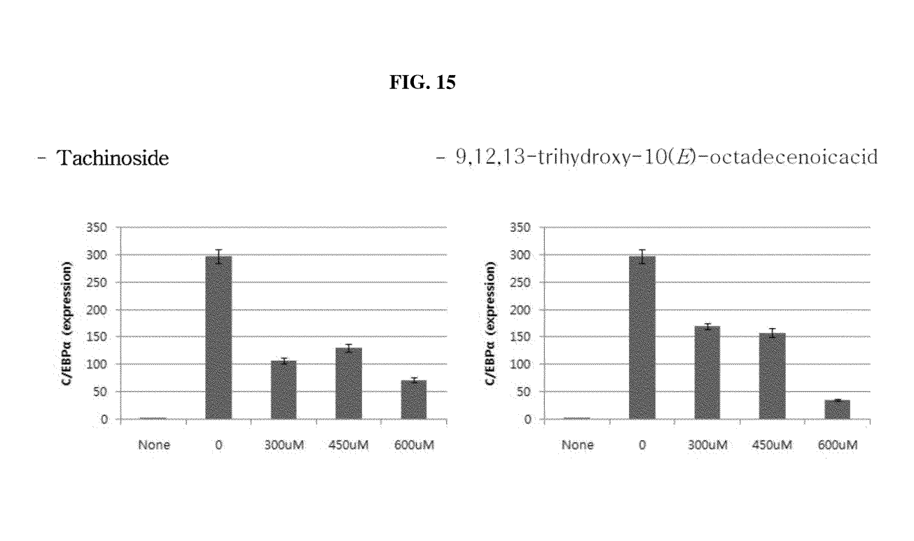

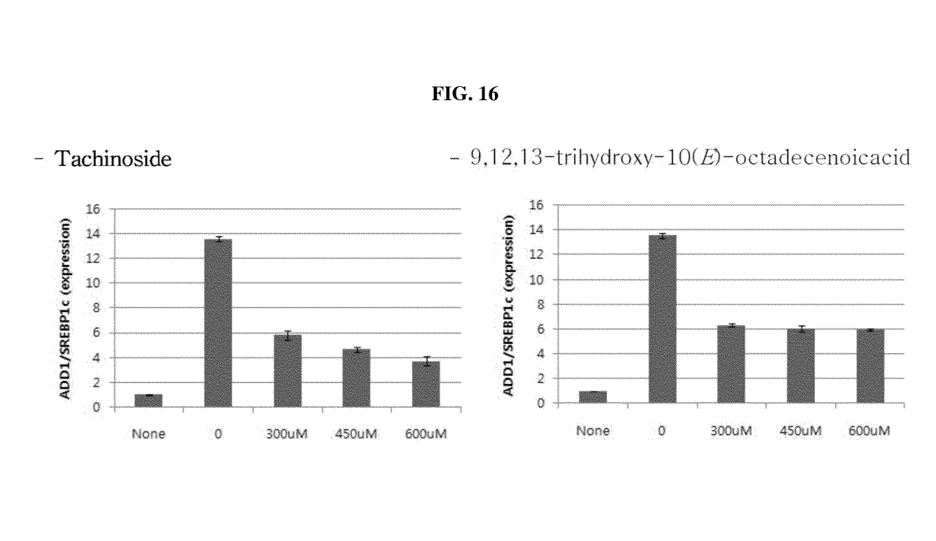

The results are shown in FIGS. 11 to 16. As can be seen in FIGS. 11 to 16, the extracts of Example 1 (Red-dog A & Red-dog B) and the compounds of Example 3 (Compounds 1 & 2) were found to inhibit the expression of PPARγ, C/EBRα and ADD1/SREBP1c in dose-dependent manners at the genetic level.

### Experimental Example 1-3-2

### Inhibitory Activity Assay at Protein Level

After the 313-L1 cells were treated with the samples (Red-dog A & Red-dog B, Compounds 1 & 2) at regular intervals of two days in the same manner as in the adipocyte differentiation method, they were washed twice with PBS and lyzed in RIPA buffer (50 mM Tris-HCl, pH 8.0, 150 mM sodium chloride, 1% NP-40, 0.5% sodium dexycholate, 0.1% sodium dodecyl sulfate, protease inhibitor). Proteins harvested by centrifugation at 13,000 rpm for 30 min were separated on 8% SDS-PAGE gel and then transferred onto a membrane. The membrane was blocked with TBST containing 5% skim milk and reacted with a primary antibody (PPARγ, C/EBPα) (Santa Cruz) and then with a secondary antibody (Santa Cruz), followed by color development with an ECL reagent (Thermo scientific) to compare the expression level of PPARγ and C/EBPα with that of β-actin.

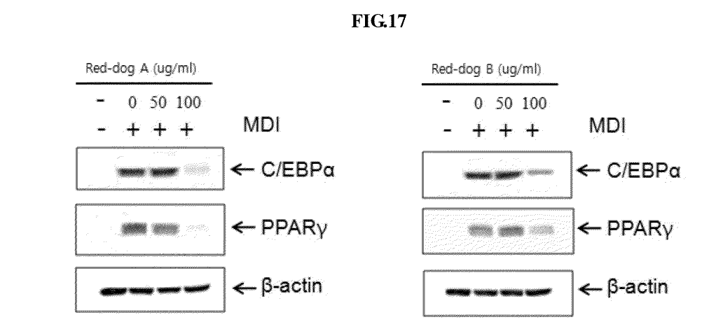

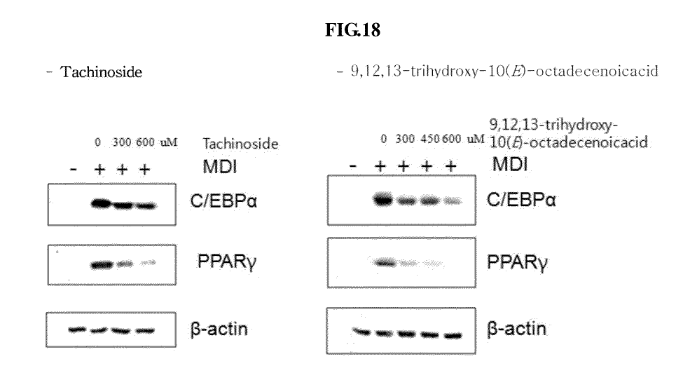

The results are shown in FIGS. 17 and 18.

Similar to the data of FIGS. 11 to 16, the extracts of Example 1 (Red-dog A & Red-dog B) and the compounds of Example 3 (Compounds 1 & 2) inhibited the expression of PPARγ and C/EBRα in dose-dependent manners.

### Experimental Example 2

### Assay for Anti-Insulin Resistance Activity

To examine whether the samples (Red-dog A & Red-dog B, compounds 1 & 2) are effective as anti-insulin resistance agents, expression levels of resistin, aP2 and Fas in the cells that had been differentiated for 8 days as in Experimental Example 1 were analyzed using the primers of Table 2, below.

TABLE 2
Primers

| Gene name | Forward primer | Reverse primer |
|---|---|---|
| GAPDH | GAGTCAACGGATTTG | GACAAGCTTCCCGTT |
| | GTCGT | CTCAG |
| | (SEQ ID NO: 9) | (SEQ ID NO: 10) |
| aP2 | CATGGCCAAGCCCAA | CGCCCAGTTTGAAGG |
| | CAT | AAATC |
| | (SEQ ID NO: 11) | (SEQ ID NO: 12) |
| Resistin | TCAACTCCCTGTTTCCA | TCTTCACGAATGTCC |
| | AATGC | CACGA |
| | (SEQ ID NO: 13) | (SEQ ID NO: 14) |
| Fas | CTGAGATCCCAGCACTT | GCCTCCGAAGCCAAA |
| | CTTGA | TGAG |
| | (SEQ ID NO: 15) | (SEQ ID NO: 16) |

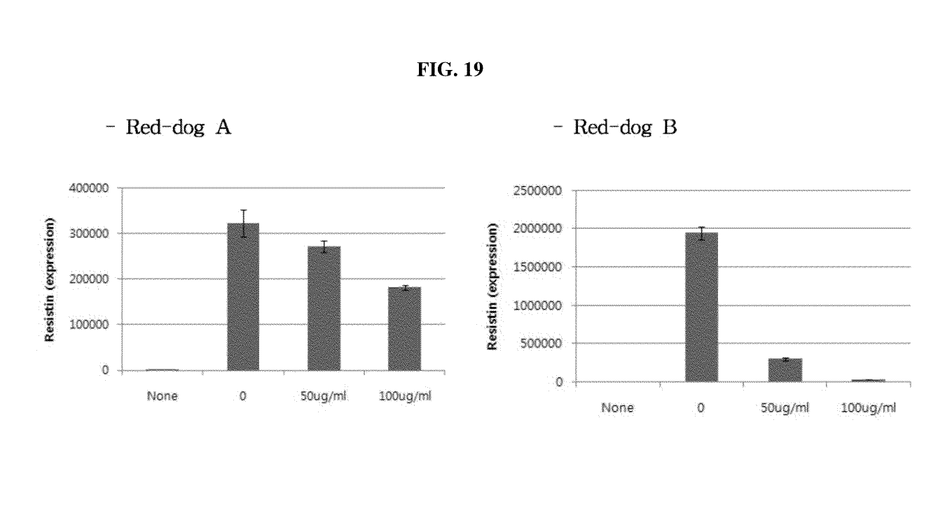

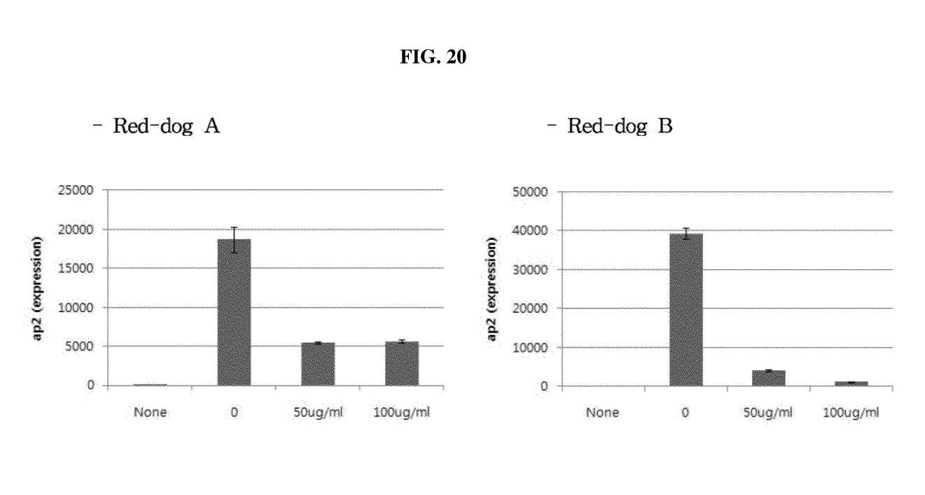

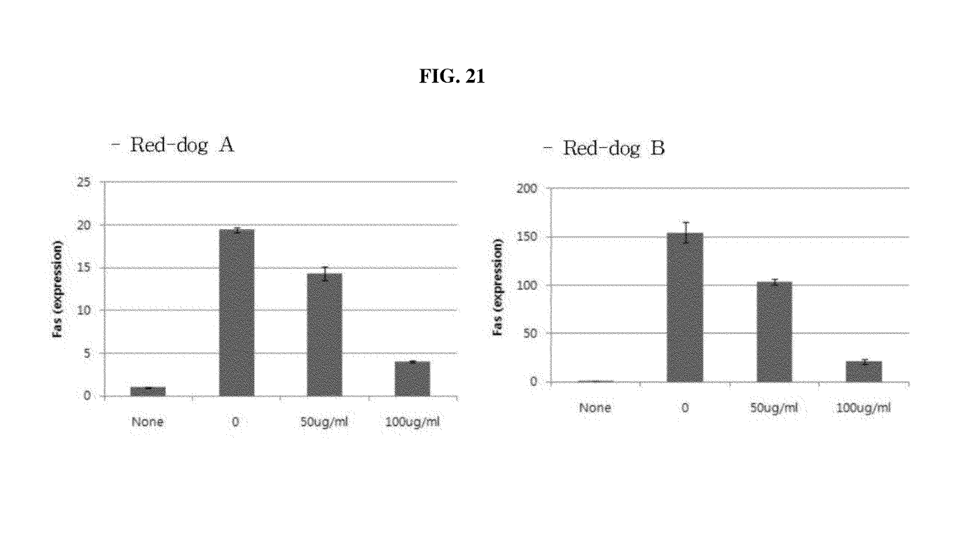

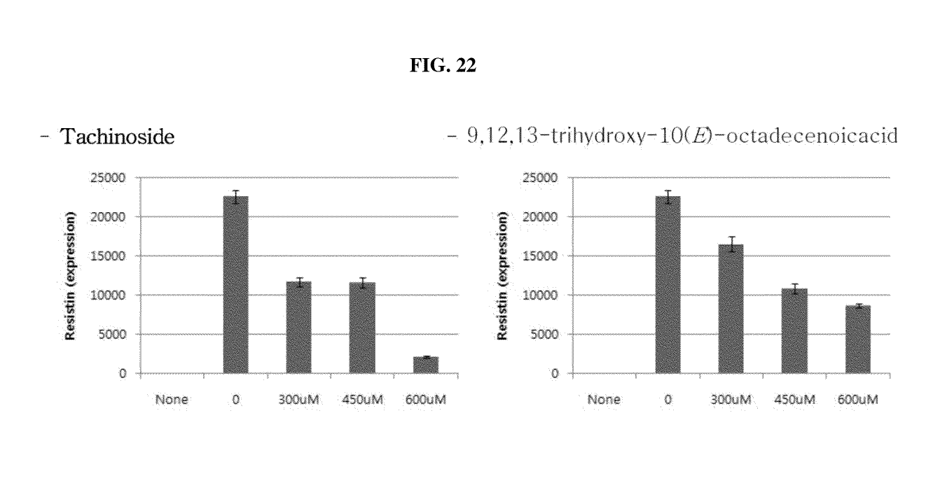

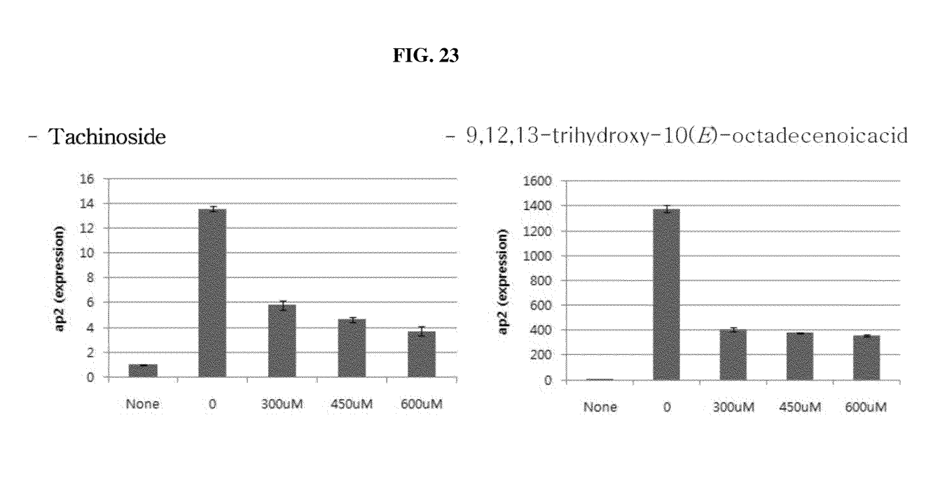

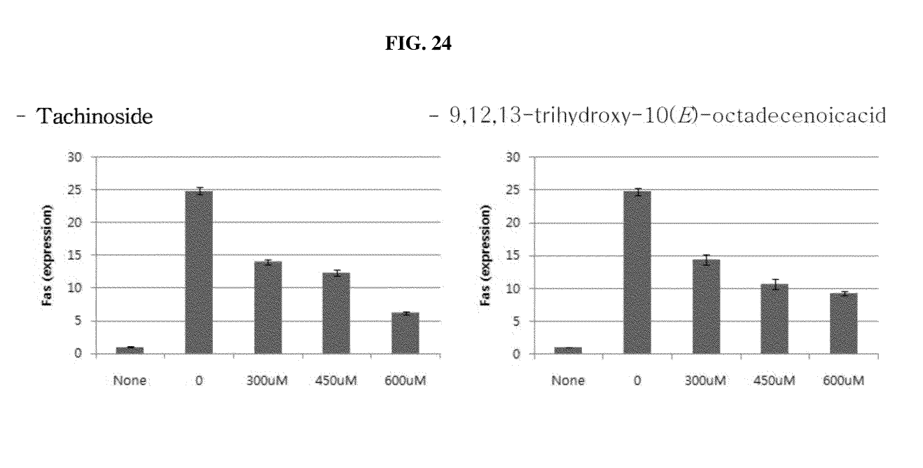

The results are shown in FIGS. 19 to 24. All the extracts of Example 1 (Red-dog A & Red-dog B) and the compounds of Example 3 (Compounds 1 & 2) were found to inhibit the expression of resistin, aP2 and Fas in dose-dependent manners at the genetic level.

What is claimed is:

1. A composition for improving obesity, comprising a wheat bran extract, tachioside, or 9,12,13-trihydroxy-10(E)-octadecenoic acid as an active ingredient.

2. The composition of claim 1, wherein the wheat bran extract is obtained by extraction with water, ethanol or a mixture thereof.

3. The composition of claim 3, being in a form of a food composition.

4. The composition of claim 1, being in a form of a pharmaceutical composition.

5. A composition for dieting, comprising a wheat bran extract, tachioside, or 9,12,13-trihydroxy-10(E)-octadecenoic acid as an active ingredient.

6. The composition of claim 5, wherein the wheat bran extract is obtained by extraction with water, ethanol or a mixture thereof.

7. The composition of claim 5, being in a form of a food composition.

8. The composition of claim 5, being in a form of a pharmaceutical composition.

9. A composition for anti-insulin resistance, comprising a wheat bran extract, tachioside, or 9,12,13-trihydroxy-10(E)-octadecenoic acid as an active ingredient.

10. The composition of claim 9, wherein the wheat bran extract is obtained by extraction with water, ethanol or a mixture thereof.

11. The composition of claim 9, being in a form of a food composition.

12. The composition of claim 9, being in a form of a pharmaceutical composition.


SEQUENCE LISTING (16 sequences)

SEQ ID NO: 1 - DNA, 20 bp, Artificial Sequence
  Primer
gagtcaacggatttggtcgt

SEQ ID NO: 2 - DNA, 20 bp, Artificial Sequence
  primer
gacaagcttcccgttctcag

SEQ ID NO: 3 - DNA, 20 bp, Artificial Sequence
  primer
cgctgatgcactgcctatga

SEQ ID NO: 4 - DNA, 22 bp, Artificial Sequence
  primer
agaggtccacagagctgattcc

SEQ ID NO: 5 - DNA, 20 bp, Artificial Sequence
  primer
aggtgctggagttgaccagt

SEQ ID NO: 6 - DNA, 20 bp, Artificial Sequence
  primer
cagcctagagatccagcgac

SEQ ID NO: 7 - DNA, 20 bp, Artificial Sequence
  primer
caaactgcccatccaccgac

SEQ ID NO: 8 - DNA, 20 bp, Artificial Sequence
  primer
tgcctcctccactgccacaa

SEQ ID NO: 9 - DNA, 20 bp, Artificial Sequence
  primer
gagtcaacggatttggtcgt

SEQ ID NO: 10 - DNA, 20 bp, Artificial Sequence
  primer
gacaagcttcccgttctcag

SEQ ID NO: 11 - DNA, 18 bp, Artificial Sequence
  primer
catggccaagcccaacat

SEQ ID NO: 12 - DNA, 20 bp, Artificial Sequence
  primer
cgcccagtttgaaggaaatc

SEQ ID NO: 13 - DNA, 22 bp, Artificial Sequence
  primer
tcaactccctgtttccaaatgc

SEQ ID NO: 14 - DNA, 20 bp, Artificial Sequence
  primer
tcttcacgaatgtcccacga

SEQ ID NO: 15 - DNA, 22 bp, Artificial Sequence
  primer
ctgagatcccagcacttcttga

SEQ ID NO: 16 - DNA, 19 bp, Artificial Sequence
  primer
gcctccgaagccaaatgag